(19) United States
(12) Patent Application Publication
(10) Pub. No.: US 2016/0058947 A1
(43) Pub. Date: Mar. 3, 2016

(54) SYRINGE FOR DISPENSING A MEDICAMENT

(71) Applicant: 3M INNOVATIVE PROPERTIES COMPANY, Saint Paul, MN (US)

(72) Inventors: Marc Peuke, Schöndorf (DE); Andreas J. Boehm, Reichling (DE)

(21) Appl. No.: 14/785,022

(22) PCT Filed: Apr. 24, 2014
(86) PCT No.: PCT/US2014/035232

§ 371 Date: Oct. 16, 2015

(30) Foreign Application Priority Data
Apr. 29, 2013 (EP) 13165735.5

(51) Int. Cl. A61M 5/24

(52) U.S. Cl. CPC A61M 5/2448

(57) ABSTRACT

A syringe for dispensing a medicament comprises a cartridge, a plug and an activator member. The syringe comprises a main chamber containing a first liquid and an auxiliary chamber containing a second liquid. To activate the syringe and merging the first and second liquid to form the medicament the auxiliary chamber and the main chamber are connectable for fluid communication with each other. A movement of the activator member and/or the plug causes the second liquid to be transferred from the auxiliary chamber into the main chamber. The syringe helps facilitating the preparation of a medicament that has to be mixed from different liquids shortly prior to use.

### FIELD OF THE INVENTION

The invention relates to a syringe for dispensing a medicament which can be mixed within the syringe from at least two liquids. In particular the invention relates to a syringe which has a main chamber containing a first liquid and an auxiliary chamber containing the second liquid. The syringe is adapted to connect the main chamber and the auxiliary chamber for fluid communication and for transferring the second liquid from the auxiliary chamber into the main chamber with the first liquid.

### BACKGROUND ART

There are medicaments which are prepared by mixture from at least two components only short before use. For example local anesthesia are often provided as a liquid prefilled in a syringe or cartridge and, prior to use, a buffer solution is added to the liquid local anesthesia, for example to obtain a desired pH value of the mixture. Typically a doctor, for example a dentist, manually mixes local anesthesia and the buffer by dispensing both liquids into a common container and aspirating both liquids back into the syringe.

There are further syringes which facilitate mixing of an anesthesia and a buffer. For example U.S. 2009/292271 A1 discloses a device for delivering a buffer solution into a buffer cartridge that comprises a needle assembly and a pusher. The septa of a buffer cartridge and an anesthetic cartridge are advanced onto a transfer needle, and the pusher advances a plunger into the buffer cartridge to deliver buffer through the transfer needle into the anesthetic cartridge. A separate exhaust needle allows excess anesthetic from the anesthetic cartridge to be exhausted. A compression member is usually provided to maintain a force against the plunger on the buffer cartridge to pressurize and stabilize the contents during sterilization and/or storage. Although existing syringes provide useful advantages there is still a need for a syringe which is easy to use and relatively inexpensive.

### SUMMARY OF THE INVENTION

In one aspect the invention relates to a syringe for dispensing a medicament. The syringe comprises:
a cartridge forming a main chamber and having a first tubular portion extending along a longitudinal axis and being in fluid communication with the main chamber;
a plug forming an auxiliary chamber;
the plug comprising an activator member which is movable to activate the syringe by causing a first liquid and a second liquid to merge to form the medicament;
the plug being received within the first tubular portion for sealing the main chamber;
the so sealed main chamber being substantially air free filled with the first liquid and the auxiliary chamber containing the second liquid;
the plug being movable within the first tubular portion in at least a first direction along the longitudinal axis for extruding at least part of the medicament;
the activator member being movable in the first direction to cause the auxiliary chamber and the main chamber to connect for fluid communication with each other such that the first and second liquid can merge; and
wherein the syringe is adapted such that a further movement of the activator member toward the first direction causes at least part of the second liquid to be transferred from the auxiliary chamber into the main chamber and thereby the plug to displace within the first tubular portion in a second direction opposite to the first direction.

The invention is advantageous in that it allows a preparation of a medicament mixed form two or more components at a minimized number of preparation steps. Further the invention helps maximizing the reliability in the preparation of a medicament in that the syringe of the invention provides the correct combination and quantities of the components. The risk of mixing tolerances or false combinations of components thus is minimized Further the invention is advantageous in that the preparation of the medicament by use of a syringe of the invention can be performed at a minimized amount of time.

In one embodiment the plug has a second tubular portion extending along the longitudinal axis and being in fluid communication with the auxiliary chamber. The activator member may be slidably received within the second tubular portion for sealing the auxiliary chamber. Further the activator member may be movable within the second tubular portion in the first direction for activating the syringe.

In an embodiment the cartridge has a tubular side wall which also forms the first tubular portion. Such cartridge side wall may have a substantially uniform cross-section along the longitudinal axis of the syringe. The cartridge side wall preferably directly delimits the main chamber, meaning that the first liquid is preferably in direct contact with the cartridge side wall. The cartridge side wall may at least inwardly extend at a generally uniform cross-section along the longitudinal axis. The cartridge side wall may for example extend at a circular cross-section at least inwardly, and in more particular may inwardly and outwardly extend at a circular cross-section, or, in other words may extend at a ring-shaped cross-section. The cartridge side wall thus may have an inner cartridge diameter, preferably a generally constant or uniform diameter over its length along the longitudinal axis.

In a further embodiment the plug has a front wall which separates the main chamber and the auxiliary chamber from each other. The activator member may have a piercing member, for example an elongated spike, protruding from the activator member. At a stage accommodated within the plug the piercing member further preferably protrudes toward the plug front wall. Thus the piercing member is adapted for piercing the plug front wall and connecting the auxiliary chamber and the main chamber for fluid communication. For example a movement of the activator member toward the plug front wall preferably causes the piercing member to penetrate through the plug front wall and thereby creates the connection between the main chamber and the auxiliary chamber. The auxiliary chamber may be air free or substantially air free filled with the second liquid. Due to normal incompressibility of liquids thus the activator member and the plug are hindered in a movement toward each other by the second liquid. However it has been found that urging the activator member toward the plug and thereby causing the second liquid to be pressurized results in the plug and eventually the activator member to deform so that the piercing member reaches and pierces the front wall upon reaching a certain pressure in the second liquid. In particular urging only the activator member and the cartridge toward each other first causes the front wall to be pierced and further urging only the activator member and the cartridge toward each other causes the activator member to move into the plug. Thereby the second liquid is transferred into the first liquid.

In a further embodiment the plug comprises a side wall which at least inwardly extends at a generally uniform cross-section along the longitudinal axis. The plug side wall preferably also forms the second tubular portion. Further the auxiliary chamber is preferably formed between the plug front wall, at least part of the plug side wall and the activator member.

In an embodiment the plug side wall directly delimits the auxiliary chamber, meaning that the second liquid is preferably in direct contact with the plug side wall. The plug side wall may extend at a circular cross-section at least inwardly, and in more particular may inwardly and outwardly extend at a circular cross-section, or, in other words may extend at a ring-shaped cross-section. The plug side wall thus preferably has an inner plug diameter which is smaller than the inner cartridge diameter.

In a further embodiment the plug has a deformable dome shaped rear portion. The deformable dome shaped rear portion in this embodiment preferably forms the activator member. Further the deformable dome shaped rear portion preferably forms a wall of the auxiliary chamber. The activator member is movable along the longitudinal axis by deformation of the activator member, particularly the activator member is movable along the longitudinal axis by inversion of the activator member. The activator member, for example when accommodated within the cartridge, is further preferably oriented and adapted for a movement in the first direction for activating the syringe.

In a further embodiment the plug has a front seal. The front seal preferably separates the main chamber and the auxiliary chamber from each other, and in particular preferably delimits part of the main chamber and further delimits part of the auxiliary chamber. The plug front seal is preferably adapted to rupture upon pressure exerted to the plug front seal from the auxiliary chamber such that the auxiliary chamber and the main chamber are connected for fluid communication. For example the plug front seal may be made of a plastic film or foil, optionally comprising a layer of aluminum. The plug front seal may further have a predetermined breaking point which facilitates rupturing of the seal upon exertion of a pressure. The auxiliary chamber in this embodiment thus is preferably formed between the plug front seal and the activator member, and the second liquid may be in direct contact with the plug front seal and the activator member.

In one embodiment the cartridge comprises a cartridge front having an openably sealed outlet for the medicament. The cartridge may for example be formed by a tubular body which is open on both sides and having a bottle neck on only one side. The bottle neck may be closed by a cartridge seal that can be pierced for opening. Such a cartridge may be made of glass, or of plastic material, for example. The cartridge may be adapted for use with a syringe mount having a plunger, and operating elements, like fingerplates, for manual use. The syringe mount may be used to dispense the medicament from the syringe by advancing the plug of the syringe via the plunger of the syringe mount. The main chamber thus is preferably formed between the cartridge front, the cartridge side wall and the plug. In a further embodiment the syringe comprises a pistil for mixing the first and second fluid upon shaking of the syringe.

In an alternative aspect the invention relates to a syringe for dispensing a medicament, which syringe comprises:
a cartridge forming a main chamber and an auxiliary chamber;
the cartridge having a first tubular portion extending along a longitudinal axis and being in fluid communication with the auxiliary chamber;
a plug being movable to activate the syringe by causing a first liquid and a second liquid to merge to form the medicament;
the plug being received within the first tubular portion for sealing the auxiliary chamber;
an activator member separating the main chamber and the auxiliary chamber from each other and sealing the main chamber;
the so sealed main chamber being substantially air free filled with the first liquid and the auxiliary chamber containing the second liquid;
the plug being movable within the first tubular portion in at least a first direction along the longitudinal axis for extruding at least part of the medicament;
the activator member adapted to connect the auxiliary chamber and the main chamber for fluid communication with each other such that the first and second liquid can merge; and
wherein the syringe is adapted such that a movement of the activator member toward a second direction opposite of the first direction causes at least part of the second liquid to be transferred from the auxiliary chamber into the main chamber.

In one embodiment the activator member is formed by a hollow body forming an outer ridge for sealing with an inner cartridge surface. The activator member and the plug are preferably disposed within the cartridge of the syringe. The syringe may be adapted such that a movement of the plug toward the first direction creates a pressure in the cartridge. Further the syringe is preferably adapted such that the so created pressure causes the hollow body of the activator member to compress. By compression of the activator member the ridge of the activator member preferably retracts from the inner cartridge surface. Consequently a gap is formed between the activator member and the cartridge, and thus the syringe is activated. The activator member may be resiliently suspended at the plug under pretension for a movement of the plug and the activator toward one another. Such a suspension may comprise or consist of a spring or an elastic band, for example. The activation of the syringe accordingly preferably allows, or automatically causes, the activator member to move in the second direction toward the plug by the pretension.

In a further embodiment the activator member comprises a weight for causing the activator member to move by force of gravity. Thus a user may orient the syringe after activation so that the weight urges the activator member toward the second direction. In this embodiment the resilient suspension is optional.

In one embodiment the activator member has a retraction cord which extends through the plug to an exterior of the syringe. The retraction cord may be adapted to be grasped by a user of the syringe. The retraction of the activator member via the cord preferably causes the activator to move in the second direction toward the plug. The activator member may be deformable and due to pressure exerted by the second liquid on the activator member assume a dome shape creating a gap between the cartridge and the activator member as the activator member is urged toward the second direction. The so formed gap preferably establishes the fluid communication between the auxiliary chamber and the main chamber.

### BRIEF DESCRIPTION OF THE FIGURES

FIGS. 1 to 3 show cross-sectional views of a syringe according to an embodiment of the invention at different stages of operation;

FIG. 4 shows a variant of the embodiment shown in FIGS. 1 to 3;

FIGS. 5, 6 show cross-sectional views of a syringe according to a further embodiment of the invention at different stages of operation;

FIGS. 7 to 9 show cross-sectional views of a syringe according to still a further embodiment of the invention at different stages of operation;

FIG. 10 shows a partial cross-sectional view of a syringe according to an embodiment of the invention;

FIG. 11, 12 show cross-sectional views of the syringe of FIG. 10 at different stages of operation; and

FIGS. 13, 14 show cross-sectional views of a syringe according to another embodiment of the invention at different stages of operation.

### DETAILED DESCRIPTION OF THE INVENTION

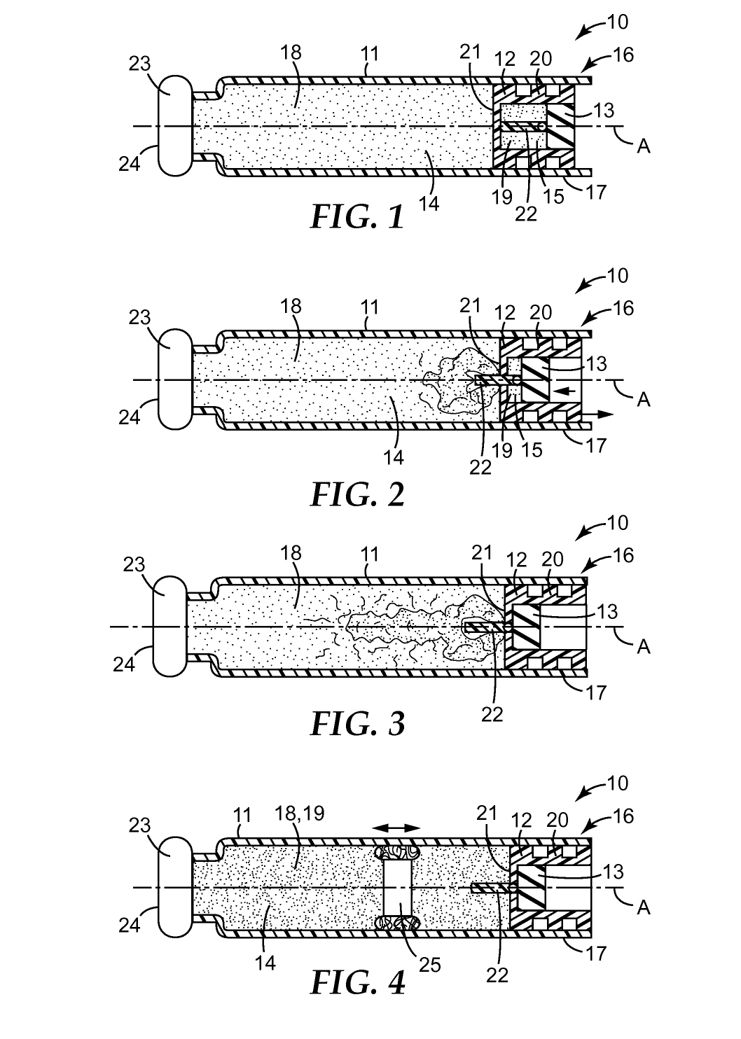

FIGS. 1 to 3 illustrate a syringe 10 for dispensing a medicament. The syringe 10 comprises a cartridge 11, a plug 12 and an activator member 13. The cartridge 11 forms a main chamber 14, and the plug 12 forms an auxiliary chamber 15. A first liquid 18 is contained within the main chamber 14, and a second liquid 19 is contained in the auxiliary chamber 15. The main chamber 14 is sealed by the plug 12 at a rear end 16 of the cartridge 11. The cartridge 11 in the example has a substantially tubular wall laterally delimiting the main chamber 14, and particularly at the rear end 16 the cartridge 11 has a first tubular portion 17 which extends along a longitudinal axis A of the syringe 10. Accordingly the first tubular portion 17 is in fluid communication with the main chamber 14. The plug 12 is received within the first tubular portion 17 and thus seals the main chamber 14.

The plug 12 receives the activator member 13. The activator member 13 is movable in a first direction (in the Figure from the right toward the left) to activate the syringe 10 by causing the first liquid 18 and the second liquid 19 to merge. In particular in the example the plug 12 has a second tubular portion 20 that extends along the longitudinal axis A and being in fluid communication with the auxiliary chamber 15. The activator member 13 is slidably received within the second tubular portion 20 for sealing the auxiliary chamber 15. Further the activator member 13 is movable within the second tubular portion 20 in the first direction for activating the syringe. The plug 12 has a front wall 21 which separates the main chamber 14 and the auxiliary chamber 15 from each other. Further the activator member 13 has a piercing member 22 that protrudes from the activator member toward the plug front wall 21 for piercing the plug front wall 21 upon movement of the activator member 13 in the first direction. Thus the auxiliary chamber 15 and the main chamber 14 can be connected for fluid communication such that the first and second liquid 18, 19 can merge to form the medicament.

As illustrated in FIG. 2 the syringe is adapted such that a further movement of the activator member 13 toward the first direction causes at least part of the second liquid 19 to be transferred from the auxiliary chamber 15 into the main chamber 14. Thereby the plug 12 displaces within the first tubular portion 17 in a second direction (in the Figure from the left toward the right) opposite to the first direction. Because the main chamber 14 is substantially air free filled with the first liquid 18, any transfer of the second liquid 19 from the auxiliary chamber 15 toward the main chamber 14 forces the plug 12 to move and thus to provide the volume required for the merged first and second liquid 18, 19. It has been found that urging the activator member 13 toward the first direction against the cartridge 11 without urging the plug 12 in the same direction causes the plug 12 to move opposite of the first direction.

Once substantially all of the second liquid 19 is transferred into the main chamber 14, as illustrated in FIG. 3, the plug 12 may be moved within cartridge 11 in the first direction along the longitudinal axis A for extruding the medicament. The cartridge 11 at front end 23 preferably has an openably sealed outlet 24 for the medicament. The skilled person will recognize different embodiments of seals that are openable, for example by a user. In a preferred embodiment the cartridge 11 has a seal film that can be pierced open, for example by an injection needle. Preferably the cartridge 11 is made of a glass or plastic material, and the plug 12 and the seal film may be made of a resilient plastic or rubber material. The activator member 13 (and the piercing member 22) may be made of a comparatively rigid plastic material or a metal.

FIG. 4 shows the syringe 10 which has a pistil 25 movably disposed within the cartridge 11. The pistil 25 is of a ring-shaped cross-section and by movement causes the liquids 18, 19 to flow through the opening left within the ring-shape. Therefore the liquids 18, 19 are agitated so that they mix with one another. The pistil 25 may accordingly be used to mix the first and second liquid 18. 19 once they are merged within the main chamber 14.

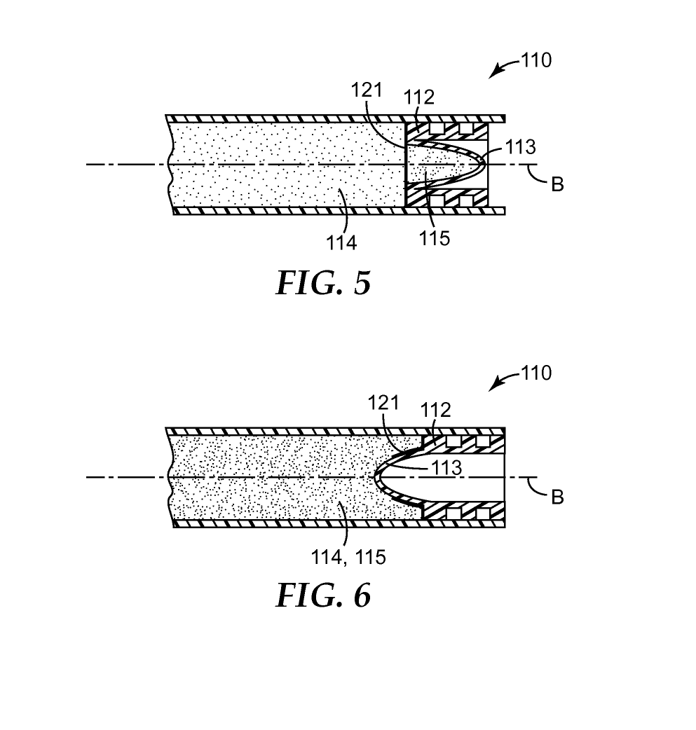

FIG. 5 shows a syringe 110 which is identical to the syringe 10 except for the plug configuration. The syringe 110 has a plug 112 which has a deformable dome shaped rear portion forming the activator member 113. The activator member 113 also forms a wall of an auxiliary chamber 115. In the example shown the activator member 113 is movable in a first direction (in the Figure from the right toward the left) along a longitudinal axis B of the syringe 110 by deformation of the activator member 113. In particular the activator member 113 may be inverted as illustrated in FIG. 6 for activating the syringe.

The plug 112 has a front seal 121 which separates the main chamber 114 and the auxiliary chamber 115 from each other. The front seal 121 is adapted to rupture upon pressure exerted to the front seal from the auxiliary chamber 115. For example the front seal 121 may be formed from a relatively thin film or foil. The front seal 121 may further comprise one or more predetermined breaking zones or lines. Thus a controlled rupturing of the front seal 121 may be facilitated. Upon rupturing of the front seal the auxiliary chamber 115 and the main chamber 114 are connected for fluid communication. In this example the auxiliary chamber 114 is formed between the plug front seal 121 and the activator member 113 only.

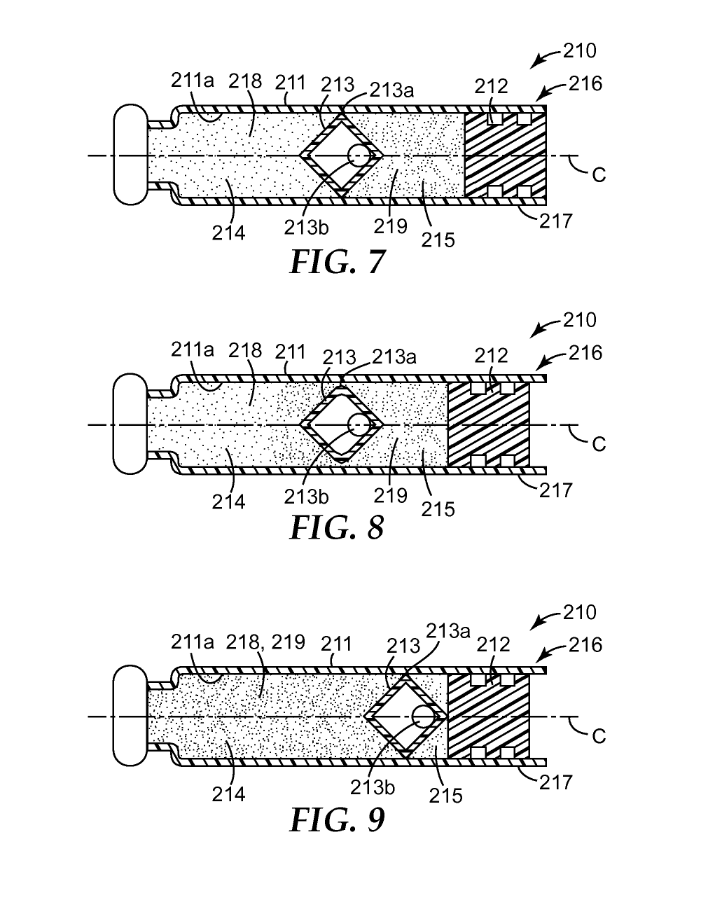

FIG. 7 shows a syringe 210 for dispensing a medicament. The syringe 210 comprises a cartridge 211 forming a main chamber 214 and an auxiliary chamber 215. The main chamber 214 comprises a first liquid and the auxiliary chamber 215 comprises a second liquid 219, the first and second liquid being adapted to form the medicament. The cartridge 211 generally has a tubular shape and in particular has at a rear end 216 a first tubular portion 217. The tubular portion 217 extends along a longitudinal axis C and is in fluid communication with the auxiliary chamber 215. The syringe 210 further comprises a plug 212 which is movable to activate the syringe by causing the first liquid 218 and the second liquid 219 to merge to form the medicament. The plug 212 is received within the first tubular portion for sealing the auxiliary chamber 215.

The syringe 210 further has an activator member 213 that separates the main chamber 214 and the auxiliary chamber 215 from each other. Further the activator member 213 seals the main chamber 214. In the example shown the activator member 213 is formed of a hollow body. The hollow body is formed of walls having a shape of a double cone connecting to each other at their wide ends. The hollow body is preferably made of a resilient material, for example rubber, and may contain a gas (for example air) hermetically sealed within and by the walls of the hollow body. At the area at which the cones connect to a double cone a sealing ridge 213a is formed. Accordingly the activator member 213 seals the main chamber 214 due to the ridge 213a sealing with an inner surface 211a of the cartridge 211. Optionally the gas within the activator member 213 may have an overpressure relative to the normal atmospheric pressure (or the pressure outside of the syringe 210) to maximize the sealing effect. The so sealed main chamber 114 is substantially air free filled with the first liquid 218.

The plug 212 is movable within the first tubular portion 217 in at least a first direction (in the Figure from the right toward the left) along the longitudinal axis C for extruding at least part of the medicament. A movement of the plug 212 further creates a pressure within the auxiliary chamber 214 and the main chamber 215 and consequently causes the activator member 213 to shrink. Thus upon reaching a certain pressure in the auxiliary chamber 214 and the main chamber 215 the sealing ridge 213a retracts from the inner surface 211a of the cartridge (shown in FIG. 8) so that the auxiliary chamber 214 and the main chamber 215 become connected for fluid communication with each other. Thus the first and second liquid 218, 219 can merge.

The syringe 210 is further adapted such that a movement of the activator member 213 toward a second direction opposite of the first direction causes at least part of the second liquid 219 to be transferred from the auxiliary chamber 215 into the main chamber 214 (shown in FIG. 9). In the example the activator member 213 comprises a weight 213b. A user may thus by appropriate orientation of the syringe 210 cause the activator member 213 to move toward the second direction (toward the plug 212) by force of gravity. In the Figure the syringe 210 may for example be oriented with its longitudinal axis C generally vertically and with the plug 212 oriented downwards. Upon exerting a pressure onto the liquids 218, 219 by movement of the plug 212 into the cartridge 211 the activator member 213 preferably shrinks and the weight 213b urges the activator downwards toward the plug 212. Thereby the second liquid 219 is transferred from the auxiliary chamber 215 into the main chamber 214. The movement of the activator member 213 thus causes the auxiliary member to reduce in volume and the main chamber to increase in volume. Thus the second liquid 219 bypasses the activator member 213 into the main chamber 214 via a gap between the cartridge 211 and the activator member 213.

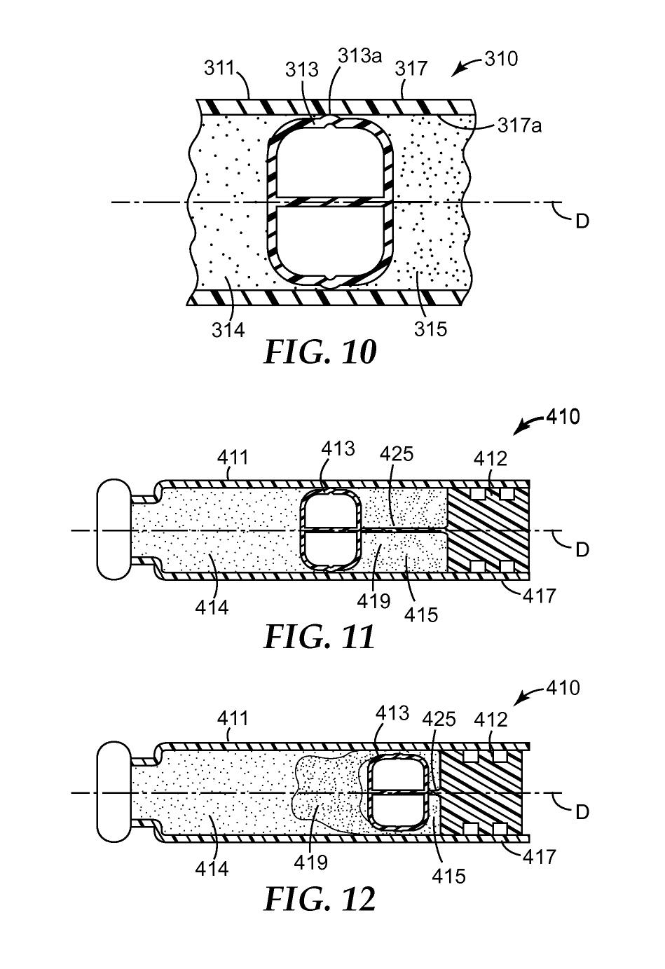

FIG. 10 shows a syringe 310 having (compared to the embodiment shown in FIGS. 7 to 9) an alternative activator member 313. The activator member 313 is generally cylindrical and having a circumferential bulge 313a forming a diameter maximum of the activator member 313 at about half of the cylinder length. The diameter maximum is dimensioned such that the activator member 313 seals with an inner surface 317a of a tubular portion 317 of the cartridge 311. In the example at least the portion of the activator member 313 forming the bulge is made of a resilient material, which thus upon creating a pressure in the main chamber 314 and/or the auxiliary chamber 115 is adapted to collapse. Accordingly the auxiliary chamber 315 and the main chamber 314 are connected for fluid communication upon pressurizing the activator member 313. The shape of the activator member 313 preferably prevents the activator member 313 from inadequate canting, for example from turning in a position in which a gap is formed between the bulge 313a of the activator member 313 and the cartridge 311. The skilled person will recognize that instead of an ideal the cylinder shape a barrel shape, or similar or equivalent shape may be used for the activator member 313.

FIG. 11 shows a syringe 410 having an activator member 413 which is identical with the activator member 313 of FIG. 10. In the example the activator member 413 is however connected to a plug 412 via a spring 425 that is held under pretension by a liquid 419 filled with auxiliary chamber 415. Because the activator member 413 seals with the cartridge 411 the liquid 419 cannot escape from the auxiliary chamber 415 although it is pressurized by the spring 425. However upon moving the plug 412 to create a (further) pressure in the auxiliary chamber 415 and/or main chamber 414 a gap is formed between the activator member 413 and the cartridge 411 so that the liquid 419 can bypass the activator member 413. Hence at that stage the activator member 413 is automatically retracted by the spring 425 causing also the liquid 419 to transfer into the main chamber 414 (shown in FIG. 12).

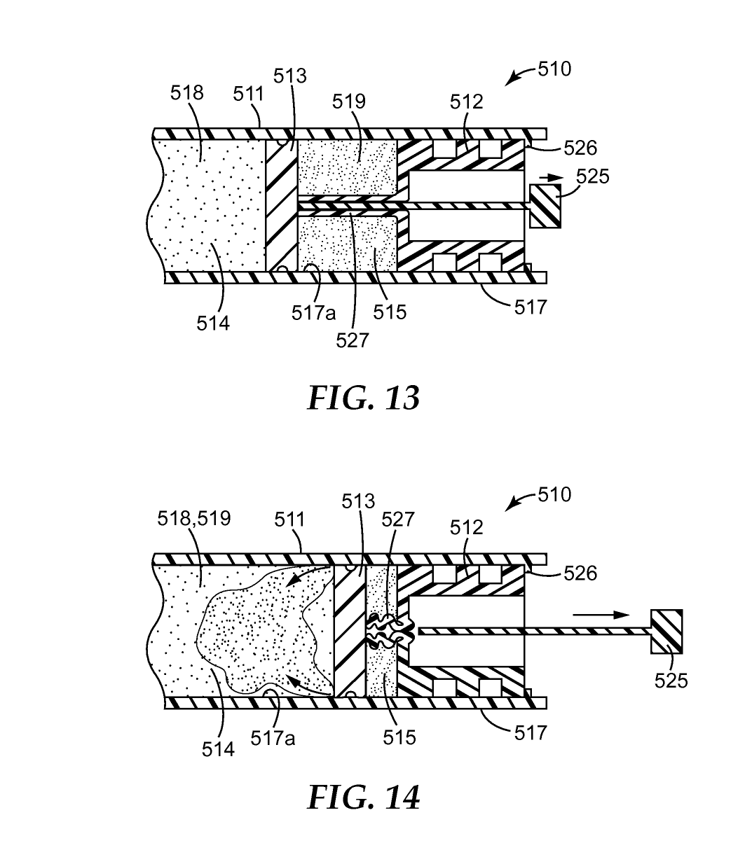

FIG. 13 shows a syringe 510 having an activator member 513 and a plug 512 which are disposed spaced from each other within a tubular portion 517 of a cartridge 511. The syringe 510 is adapted such that the plug 513 is movable in a first direction (in the Figure from the right toward the left) for dispensing a medicament contained within the syringe 510 and merged within the syringe from separately stored first and second liquids 518, 519. The space delimited by the activator member 513 the plug 512 and the cartridge 511 forms an auxiliary chamber 515 in which the second liquid 519 is contained. The first liquid 518 is contained in a main chamber 514 formed by a continuation of the tubular portion 517 of the cartridge 511. The activator member 513 in the example has a retraction cord 525 which protrudes from the activator member through the plug 512 to an exterior of the syringe 510. Thus a user can grasp the retraction cord 525 for pulling back the activator member 513 relative to the plug 512 in a second direction opposite of the first direction. To avoid retraction of the plug 512 together with the retraction of the activator member 513 the plug 512 is preferably held in place during retraction of the activator member 513. For this purpose the cartridge 511 may have a retainer 526 which prevents a movement of the plug 512 in the second direction.

The activator member 513 is adapted to seal with an inner surface of the cartridge 511, particularly with an inner surface 517a of the tubular portion 517. Further the activator member 513 may be generally disk shaped, for example having a thickness that is smaller than its diameter, and may be made of a resilient material. Thus the activator member 513 is adapted to deform toward a dome shape upon retraction by the retraction cord against pressure built up in the auxiliary chamber 515 in the second liquid 519. Such pressure acts upon the circumferential portion of the activator member 513 and thus causes the circumferential portion of the activator member 513 to bend away from that pressure, thus preferably assuming the dome shape. At some point as a certain pressure is reached the dome shape results in the diameter of the activator member 513 to become smaller than the diameter of the inner surface 517a of the cartridge so that a gap is created between the activator member 513 and the cartridge. Accordingly a fluid communication is established between the main chamber 514 and the auxiliary chamber 515 via the gap. A further movement of the activator member 513 toward the second direction finally results in the second liquid 519 to be transferred from the auxiliary chamber 515 into the main chamber 514 where it merges with the first liquid 518 to form the medicament (shown in FIG. 14). In the example the activator member 513 and the plug 512 are additionally connected to each other by a sleeve 527 within which the retraction cord is accommodated. Thus the retraction cord 525 is prevented from getting in contact with the second liquid 519. Accordingly also a user may be prevented from getting in contact with the second liquid 519 when the retraction cord 525 is retracted out of the syringe 510. This is particularly advantageous in an embodiment in which the retraction cord 525 is adapted to tear off after activation of the syringe 510 as indicated in FIG. 14.

1. A syringe for dispensing a medicament, comprising:
a cartridge forming a main chamber and having a first tubular portion extending along a longitudinal axis and being in fluid communication with the main chamber;
a plug forming an auxiliary chamber;
the plug comprising an activator member which is movable to activate the syringe by causing a first liquid and a second liquid to merge to form the medicament;
the plug being received within the first tubular portion for sealing the main chamber;
the so sealed main chamber being substantially air free filled with the first liquid and the auxiliary chamber containing the second liquid;
the plug being movable within the first tubular portion in at least a first direction along the longitudinal axis for extruding at least part of the medicament; and
the activator member being movable in the first direction to cause the auxiliary chamber and the main chamber to connect for fluid communication with each other such that the first and second liquid can merge;
wherein the syringe is adapted such that a further movement of the activator member toward the first direction causes at least part of the second liquid to be transferred from the auxiliary chamber into the main chamber and thereby the plug to displace within the first tubular portion in a second direction opposite to the first direction.

2. The syringe of claim 1, wherein the plug has a second tubular portion extending along the longitudinal axis and being in fluid communication with the auxiliary chamber, wherein the activator member being slidably received within the second tubular portion for sealing the auxiliary chamber, wherein the activator member is movable within the second tubular portion in the first direction for activating the syringe.

3. The syringe of claim 2, wherein the plug has a front wall which separates the main chamber and the auxiliary chamber from each other, and wherein the activator member has a piercing member protruding from the activator member toward the plug front wall for piercing the plug front wall and connecting the auxiliary chamber and the main chamber for fluid communication.

4. The syringe of claim 3, wherein the plug comprises a side wall at least inwardly extending at a generally uniform cross-section along the longitudinal axis, the plug side wall forming also the second tubular portion, and wherein the auxiliary chamber is formed between the plug front wall, at least part of the plug side wall and the activator member.

5. The syringe of claim 1, wherein the plug has a deformable dome shaped rear portion forming the activator member and forming a wall of the auxiliary chamber, the activator member being movable along the longitudinal axis by deformation, particularly inversion, of the activator member, and wherein the activator member is movable in the first direction for activating the syringe.

6. The syringe of claim 5, wherein the plug has a front seal which separates the main chamber and the auxiliary chamber from each other, and wherein the front seal is adapted to rupture upon pressure exerted to the front seal from the auxiliary chamber such that the auxiliary chamber and the main chamber are connected for fluid communication.

7. The syringe of claim 6, wherein the auxiliary chamber is formed between the plug front seal and the activator member.

8. The syringe of claim 1 any of the preceding claims, wherein the cartridge comprises a cartridge front having an openably sealed outlet for the medicament.

9. The syringe of claim 8, wherein the cartridge comprises a side wall at least inwardly extending at a generally uniform cross-section along the longitudinal axis, the cartridge side wall forming the first tubular portion, and wherein the main chamber is formed between the cartridge front, the cartridge side wall and the plug.

10. The syringe of claim 1, comprising a pistil for mixing the first and second fluid upon shaking of the syringe.

11. A syringe for dispensing a medicament, comprising:
a cartridge forming a main chamber and an auxiliary chamber;
the cartridge having a first tubular portion extending along a longitudinal axis and being in fluid communication with the auxiliary chamber;
a plug being movable to activate the syringe by causing a first liquid and a second liquid to merge to form the medicament;
the plug being received within the first tubular portion for sealing the auxiliary chamber;
an activator member separating the main chamber and the auxiliary chamber from each other and sealing the main chamber;
the so sealed main chamber being substantially air free filled with the first liquid and the auxiliary chamber containing the second liquid;
the plug being movable within the first tubular portion in at least a first direction along the longitudinal axis for extruding at least part of the medicament; and
the activator member adapted to connect the auxiliary chamber and the main chamber for fluid communication with each other such that the first and second liquid can merge;
wherein the syringe is adapted such that a movement of the activator member toward a second direction opposite of the first direction causes at least part of the second liquid to be transferred from the auxiliary chamber into the main chamber.

12. The syringe of claim 11, wherein the activator member is formed by a hollow body forming an outer ridge for sealing with an inner cartridge surface, wherein the syringe is adapted such that a movement of the plug toward the first direction by creation of a pressure in the cartridge causes the hollow body of the activator member to compress, thereby the ridge to retract from the inner cartridge surface and thus the syringe to activate.

13. The syringe of claim 12, wherein the activator member is resiliently suspended at the plug under pretension for a movement of the plug and the activator toward one another, and wherein activation of the syringe allows the activator member to move in the second direction toward the plug by the pretension.

14. The syringe of claim 12, wherein the activator member comprises a weight for causing the activator member to move by force of gravity.

15. The syringe of claim 11, wherein the activator member has a retraction cord extending through the plug to an exterior of the syringe, the retraction cord being adapted to be grasped by a user of the syringe, and wherein retraction of the activator member via the cord causes the activator to move in the second direction toward the plug.
